(19) United States
(12) Patent Application Publication
(10) Pub. No.: US 2009/0088676 A1
(43) Pub. Date: Apr. 2, 2009

(54) Perfusion System for Pancreas Treatment

(76) Inventor: Satoru Murata, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/182,286

(22) Filed: Jul. 30, 2008

(30) Foreign Application Priority Data
Jul. 31, 2007 (JP) 2007-220378

(51) Int. Cl. A61M 1/36

(52) U.S. Cl. 604/6.16

(57) ABSTRACT

A perfusion system is so formed that enclosed areas may be formed in the artery system related to the pancreas and the venous system related to the pancreas, respectively, by using balloon catheters, the blood inside the venous system in the enclosed area may be extracted outside the human body, and that, after injecting a pharmaceutical such as anticancer agent into the extracted blood, the extracted blood including pharmaceutical may be returned to the artery system in the enclosed area again.

### BACKGROUND OF THE INVENTION

The present invention relates to a perfusion system for perfusing the therapeutic agent especially for the pancreas such as anticancer agent only through the specified area of the pancreas. In this document, the term “perfusion” means such an operation that the major blood stream into the pancreas and the major blood stream from the pancreas may be occluded, and thus, the blood so isolated substantially inside the enclosed area of the pancreas may be exteriorized once, and then, the therapeutic agent, for example, such as anticancer agent, may be injected inside the exteriorized blood, and finally, the blood with the therapeutic agent injected may be returned into the body, or means such an operation that the therapeutic agent may be injected directly inside the so-formed enclosed area of the pancreas.

Next, a technical background of the present invention is described as an example in the treatment of pancreatic cancer. The pancreas is located between the stomach and the backbone, and the shape of the pancreas looks like a tadpole. The part of the pancreas corresponding to the head of the tadpole is called a pancreas head, and is located at duodenum side. The part of the pancreas corresponding to the tail of the tadpole is called a pancreas tail, and is located at spleen side. The function of the pancreas includes the secretion of important hormone such as insulin, and the creation of pancreatic fluid used as digestive fluid to be supplied through the pancreatic duct to the duodenum. Most pancreas cancer may be generated from the cells of the pancreatic duct, but the early detection of pancreatic cancer is extremely difficult and its cure after cancer detection may be too late in the present circumstances.

Though surgical treatment is generally applied as the treatment of the pancreatic cancer, it is often the case that the surgical intervention for the pancreatic cancer is extremely difficult if the pancreatic cancer is located at the area where many blood vessels are involved in a complicated pattern or metastasized at plural internal organs. Though chemotherapy using anticancer agent may be applied, the concentration of the anticancer agent cannot be increased because of its bad influence to the healthy internal organs. Thus, it is required to control the concentration of the anticancer agent as low as possible so that the cancer cell growth may be kept down, which can meet such an expectation that the growth of the cancer call may be suppressed and the cancer may be killed effectively. This means that the pancreatic cancer is one of the most critical and intractable cancers at the present days, and that its effective treatment system cannot exist. JA 2006-26368 A discloses a perfusion circuit similar to the blood perfusion circuit used with the present invention. JA 2007-21143 A and Japanese Patent Application Number 2006-357556 (2006) (unpublished reference) disclose a blood occlusion device similar to the balloon catheter used with the present invention.

### BRIEF SUMMARY OF THE INVENTION

An object of the present invention is to provide a perfusion system for pancreas treatment which enables to dose substantially the therapeutic agents for the pancreas only intensively to the pancreas and to cure the pancreas without providing bad influences over the other internal organs. For example, this object is to provide a perfusion system which enables to suppress the broth of the pancreatic cancer calls and/or kill effectively the cancer cells without abdominal operations required in stead of applying the surgical intervention inevitably accompanied with abdominal operations or applying the chemotherapy with drip infusion of the anticancer agent to be perfused all over the human body.

Now, the basic concept of the present invention is described as an example of the treatment of the pancreatic cancer. At first, as the preliminary phase, the physical arrangement and dynamics of the arterial flow provided through the coeliac artery into the pancreas and the venous flow inside the pancreas are precisely analyzed. Next, based on this analytical result, the major arterial flow and the major venous flow around the pancreas may be substantially isolated from the other internal organs, and thus, a enclosed area inside the pancreas may be formed so as to contain the cancer cells of the pancreas. Finally, the anticancer agent with dozens of concentration of the anticancer agent used by drip infusion for the whole body perfusion may be selectively applied only to the cancer cells inside the enclosed area of the pancreas by applying the anticancer agent only to the blood vessels inside the enclosed area of the pancreas. The individual equipment used in the perfusion system according to the present invention are well known in the application of the intra-pelvic perfusion system conventionally used in the treatment of the cervical cancer. Therefore, structures and operations of those equipment are not described in detail here but described briefly.

As the result of anatomical research of the blood flow inside the pancreas of pigs, it is proved that the dorsal pancreatic artery is the most important artery in order to dose the anticancer agent, because the arterial flow through the dorsal pancreatic artery reaches all over the pancreas among all the arterial flows. In addition, it is proved that the arterial flow leads to the venous flow inside the pancreas and almost all the venous flow flows out to the portal vein through the posterior superior pancreaticoduodenal vein. The perfusion system according to the present invention, which will be described in detail below, is configured according to the knowledge as described above.

Among the vascular structures of the pancreas, especially, the vascular structures of the dorsal pancreatic artery can be categorized into five patterns according to Woodburn's report in 1951 as described in Reference Document 1 (also shown in FIG. 3). The inventors of the present invention studied the portion where the cancer cells exist as well as those patterns, and researched the structure of the perfusion system for the treatment of the pancreatic cancer. As the result of the inventors' research, the inventors concluded that it is simple and effective to use balloon catheters and coils or to use micro balloon catheter used for the treatment of the brain infraction and coils in order to establish the method for forming an enclosed area described above.

As for the balloon catheter, for example, the balloon catheter as disclosed in JA 2006-26368 A or the balloon catheter as disclosed in Japanese Patent Application Number 2006-357556 is especially preferable. It will be appreciated that the method using micro balloon catheters, which can occlude relatively small-diameter blood vessels, can occlude various blood vessels more effectively in comparison with the method using normal balloon catheters. By using micro balloon catheters, it will be appreciated that the perfusion system can be configured more simply. Note that the vasoocclusive coils can be also wholly or partially replaced by micro balloon catheters.

The most general and basic perfusion system for pancreas treatment comprises; an arterial system occluding means for occluding the main blood vessels of the arterial system so that the blood in the arterial system related to the pancreas may not be substantially flow into the internal organ other than the pancreas, and forming an enclosed area in the above arterial system related to the pancreas; a venous system occluding means for occluding the main blood vessels of the venous system so that the blood in the venous system related to the pancreas may not substantially flow into the internal organ other than the pancreas, and forming an enclosed area in the above venous system related to the pancreas; a perfusion apparatus for extracting the blood inside the enclosed area of the venous system outside the human body, injecting a pharmaceutical into the extracted blood and then perfusing the venous blood into the enclosed area of the artery system; and a means for extracting the venous blood flow which is flowing into the enclosed area of the venous system outside the human body, and re-circulating the extracted venous blood flow into the human body at another part of the human body.

In the above system, the recirculation of the blood into the enclosed area of the arterial system may be performed by the arterial system occluding means, and the recirculation of the blood from the enclosed area of the venous system may be performed by the venous system occluding means. In this system, “not substantially flow into the internal organ other than the pancreas” or “not substantially flow out into the internal organ other than the pancreas” means that there is no problem for very minor amount of blood flowing in or flowing out which has no influence on another internal organ.

In the above-described perfusion system for pancreas treatment, it will be appreciated that the blood can be occluded much easier by the above arterial system occluding means configured with a single balloon catheter and plural coil apparatus, though it is allowed that the arterial system occluding means can be structured with plural micro balloon catheters.

One of more particular perfusion systems for pancreas treatment comprises: a first balloon catheter provided at a downstream side of the confluence part where the blood flow of the splenic vein flows in the portal vein so as to occlude said portal vein; a second balloon catheter provided at an upstream side of said confluence part so as to occlude the superior mesenteric vein; a third balloon catheter provided at a downstream side of the bifurcation part where the blood flow in the abdominal aorta flows into the splenic artery and at an upstream side of the bifurcation part where the blood flows from the splenic artery flows into the dorsal pancreatic artery so as to occlude the splenic artery; a first occlusion device provided at a upstream side where the splenic artery branches at the pancreatic branch of splenic artery so as to occlude the splenic artery in order to prevent the blood flow in the splenic artery from flowing into the spleen; a second occlusion device for preventing a relatively large amount of arterial flow from flowing into the stomach and the duodenum; and a perfusion apparatus for extracting the venous blood inside the human body, injecting a pharmaceutical into the extracted venous blood and then perfusion the venous blood into the human body.

In the above configuration, the venous flow occluded inside the enclosed area by above described plural occlusion device is further extracted through the first balloon catheter outside the human body, and next, after injecting a pharmaceutical into the extracted venous blood by the perfusion apparatus, the extracted venous blood is returned through the third balloon catheter again to the artery inside the enclosed area; and the blood inside the superior mesenteric vein is extracted outside the human body through the second balloon catheter at the upstream of the indwelling position of the second balloon catheter, and then the extracted blood is returned to the human body again at another position of the human body.

In the above description, “upstream” means the position where the blood flow comes from, and “downstream” means the position where the flood flow goes to. In case of arterial flow, “upstream” is the position relatively close to the heart, and “downstream” is the position relatively far from the heart. In case of venous flow, this definition should be reversed.

A particular perfusion system for pancreas treatment comprises according to another aspect of the present invention comprises: a first balloon catheter provided at a downstream side of the confluence part where the blood flow of the splenic vein flows in the portal vein so as to occlude said portal vein; a second balloon catheter provided at an upstream side of said confluence part so as to occlude the superior mesenteric vein; a third balloon catheter provided so as to occlude the dorsal pancreatic artery; a first occlusion device provided at a downstream side where the splenic artery branches at the pancreatic branch of splenic artery so as to occlude the splenic artery in order to prevent the blood flow in the splenic artery from flowing into the spleen; a second occlusion device for preventing a relatively large amount of arterial flow from flowing into the stomach and the duodenum; and a perfusion apparatus for extracting the venous blood in the human body outside the human body, and then, after injecting a pharmaceutical into the extracted venous blood, perfusing the venous blood into the human body.

In the above configuration, the venous blood occluded inside the enclosed area by the plural occlusion device is extracted through the first balloon catheter outside the human body, and then, after injecting a pharmaceutical into the extracted venous blood by the perfusion apparatus, the extracted venous blood is returned through the third balloon catheter again to the artery inside the enclosed area; and the blood inside the superior mesenteric vein is extracted outside the human body through the second balloon catheter at the upstream of the indwelling position of the second balloon catheter, and then the extracted blood is returned to the human body again at another position of the human body.

One of another embodiment of the perfusion systems for pancreas treatment comprises: a first balloon catheter provided at a downstream side of the confluence part where the blood flow of the splenic vein flows in the portal vein so as to occlude said portal vein; a second balloon catheter provided at an upstream side of said confluence part so as to occlude the superior mesenteric vein; a third balloon catheter provided at a downstream side of the bifurcation part where the blood flows in the abdominal aorta flows into the splenic artery and at an upstream side of the bifurcation part where the blood flows from the splenic artery flows into the dorsal pancreatic artery so as to occlude the splenic artery; a first occlusion device provided at a downstream side where the splenic artery branches at the pancreatic branch of splenic artery so as to occlude the splenic artery in order to prevent the blood flow in the splenic artery from flowing into the spleen; at least one, second occlusion device provided so as to occlude the blood flows in the gastroduodenal artery and the posterior superior pancreaticoduodenal artery; a pump apparatus for sucking the blood inside the human body outside the human body and for returning the sucked blood into the human body again; and a syringe apparatus for injecting the pharmaceutical into the enclosed area.

In the above configuration, the pharmaceutical is injected into the arterial flow inside the enclosed area through the third balloon catheter by the syringe apparatus; and

the blood inside the superior mesenteric vein is extracted outside the human body through the second balloon catheter at the downstream of the indwelling position of the second balloon catheter, and then the extracted blood is returned to the human body again at another position.

By using a perfusion system for pancreatic treatment according to the present invention, it will be appreciated that the treatment for pancreatic cancer and other cancer which has been recognized as a difficult problem, can be realized without abdominal operations. In addition, it will be appreciated that any undesirable side effect due to anticancer agent can be reduced as much as possible because it is not required to apply such anticancer agent as circulated all over the human body in the conventional anticancer treatment. It will be further appreciated that the perfusion system according to the present invention may be also effective to the genetic therapy and the treatment of sugar diabetes by using alternative pharmaceutical other than the pharmaceutical used for the treatment of pancreatic cancer.

### BRIEF DESCRIPTION OF THE SEVERAL VIEWS OF THE DRAWINGS

FIG. 1 is a schematic drawing showing the structure of the arterial system related to the human pancreas.

FIG. 2 is a schematic drawing showing the structure of the venous system related to the human pancreas.

FIG. 3A is a schematic drawing showing structural patterns of the human dorsal pancreatic artery.

FIG. 3B is a schematic drawing showing structural patterns of the human dorsal pancreatic artery.

FIG. 4 is a schematic drawing illustrating a method for forming an enclosed area in the venous system related to the human pancreas.

FIG. 5 is a schematic drawing illustrating a method for forming an enclosed area in the arterial system related to the human pancreas.

FIG. 6 is a schematic drawing for describing one embodiment of the present invention and illustrating an arrangement of the enclosed area formed in the arterial system related to the human pancreas and equipment such as balloon catheters.

FIG. 7 is a schematic drawing for describing another embodiment of the present invention and illustrating an arrangement of the enclosed area formed in the arterial system related to the human pancreas and equipment such as balloon catheters.

FIG. 8 is a schematic drawing for describing yet another embodiment of the present invention and illustrating an arrangement of the enclosed area formed in the arterial system related to the human pancreas and equipment such as balloon catheters.

FIG. 9 is a schematic drawing for describing yet another embodiment of the present invention and illustrating an arrangement of the enclosed area formed in the arterial system related to the human pancreas and equipment such as balloon catheters.

FIG. 10 is a schematic drawing for describing yet another embodiment of the present invention and illustrating an arrangement of the enclosed area formed in the arterial system related to the human pancreas and equipment such as balloon catheters.

FIG. 11 is a schematic drawing for describing yet another embodiment of the present invention and illustrating an arrangement of the enclosed area formed in the arterial system related to the human pancreas and equipment such as balloon catheters.

FIG. 12 is a schematic drawing for describing yet another embodiment of the present invention and illustrating an arrangement of the enclosed area formed in the arterial system related to the human pancreas and equipment such as balloon catheters.

FIG. 13A is a graph describing the result of the embodiment 1 according to the present invention.

FIG. 13B is a table showing the data for the graph describing the result of the embodiment 1 according to the present invention.

### DETAILED DESCRIPTION OF THE INVENTION

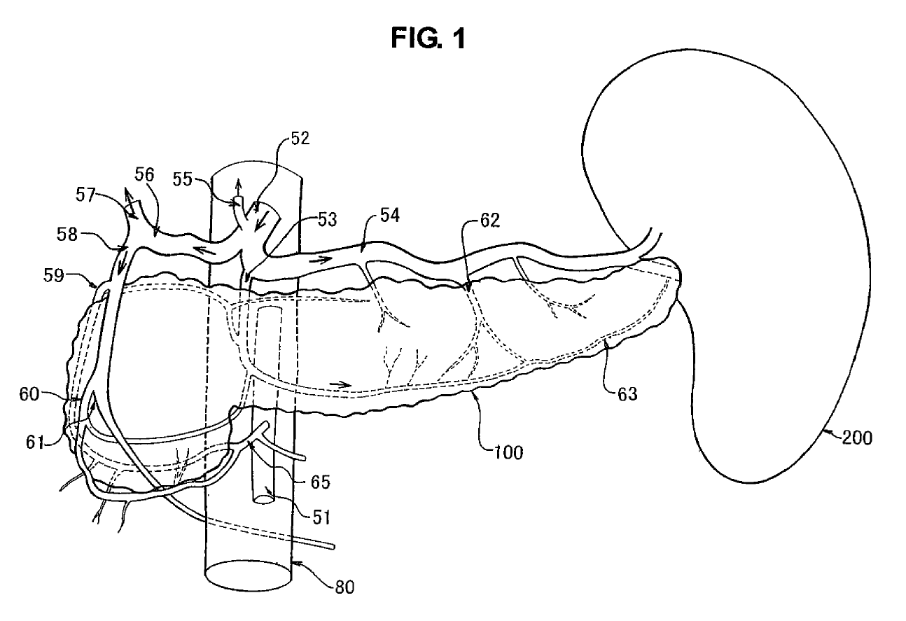

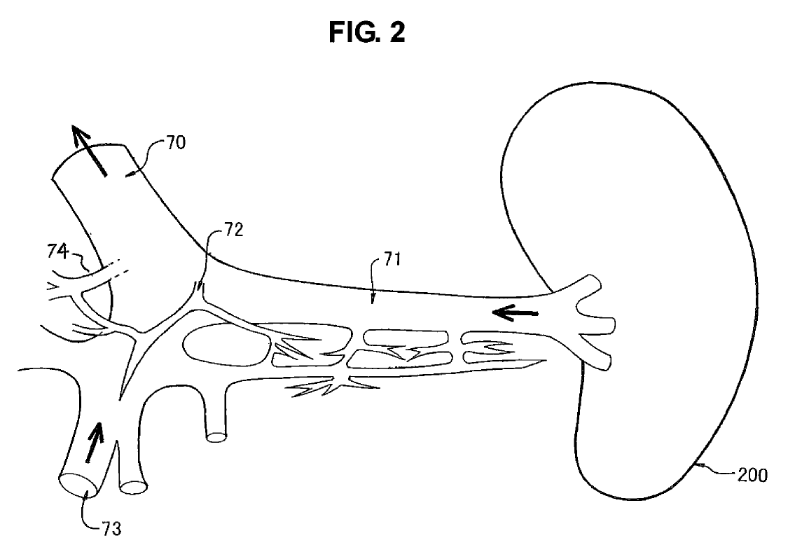

At first, the basic concept of the present invention is described by referring to FIGS. 1 to 3. FIG. 1 illustrates the outline of the arterial system related to the human pancreas. FIG. 2 illustrates the outline of the venous system related to the human pancreas. Note that an identical reference symbol represents an identical or like part in those figures.

In order to make it easier to understand the present invention, the arterial system related to the pancreas is described at first, by referring to FIG. 1 and then, the venous system related to the pancreas is described by referring to FIG. 2. In FIG. 1. the component 100 is a pancreas, the component 200 is a spleen, and the arrow represents a direction of the arterial flow. The arterial flow from the aorta 80 into the coeliac artery 52 branches and divergently flows into the splenic artery 54, the common hepatic artery 56 and the left gastric artery 55. In case of such a vascular structure as found in the human body with the highest probability, the blood flowing in the splenic artery 54 flows inside the pancreas 100 through the dorsal pancreatic artery 53, the great pancreatic artery 62 and the caudal pancreatic artery 63. The blood flowing in the common hepatic artery 56 branches and divergently flows into the proper hepatic artery 57 directing toward the liver (not shown) and the gastroduodenal artery 58 directing toward the stomach, the duodenum and the other internal organs (not shown). The blood flowing into the gastroduodenal artery 58 further flows into the posterior superior pancreaticoduodenal artery 59, the anterior superior pancreaticoduodenal artery 60 and the right gastro-omental artery 61. The arterial flow in the posterior superior pancreaticoduodenal artery 59 conflows into the inferior pancreaticoduodenal artery 65. The above-described structure is the outline of the artery system.

Next, the outline of the venous system related to the pancreas is described by referring to FIG. 2. In FIG. 2, the venous flow from the spleen 200 flows in the splenic vein 71 in the direction shown by the arrow in the figure, and then, conflows into the venous flow from the superior mesenteric vein 73, and flows to the portal vein 70. The venous flow in the pancreas flows from the dorsal pancreatic vein 72 into the splenic vein 71 as well as flows directly into the portal vein 70 through the posterior pancreatic duodenal vein 74. Note that the arterial flow flowing into the pancreas through the dorsal pancreatic artery 53 (FIG. 1) flows as a venous flow through the posterior pancreatic duodenal vein 74 into the portal vein 70. This configuration has an important meaning in the present invention. This means that it is required to form enclosed areas for the arterial system and the venous system, respectively, on the basis of the flow-in position of the dorsal pancreatic artery 53 (FIG. 1) and the flow-out position of the posterior pancreatic duodenal vein 74 in order to realize a pancreatic treatment by using an occlusive perfusion system.

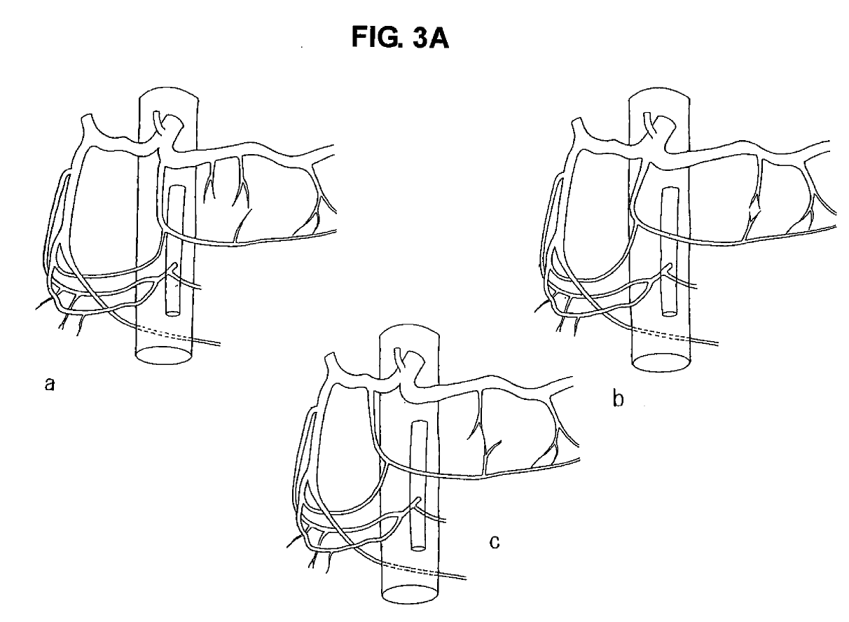

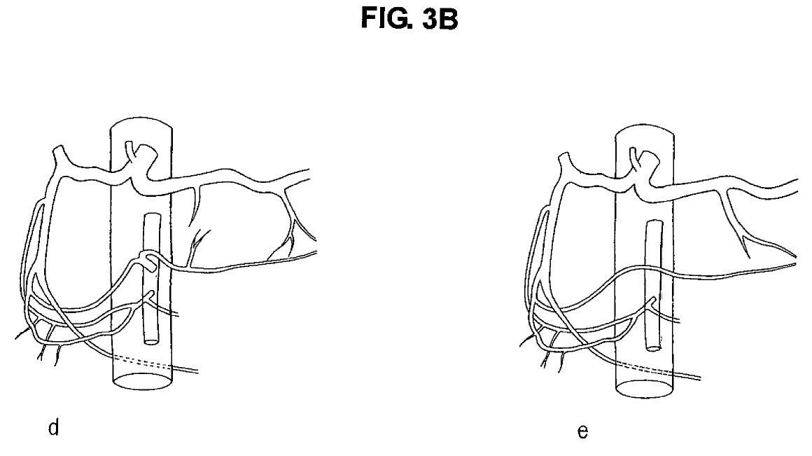

Next, refer to FIG. 3A and FIG. 3B. FIG. 3A and FIG. 3b shows various kinds of structures of the human dorsal pancreatic artery, in the order of probability found in the human body. The probability found in the human body increases in the order from (a) through (b), (d) and (d) to (e) in FIG. 3A and FIG. 3B. FIG. 3A(a) shows the case that the dorsal pancreatic artery branches from the splenic artery, FIG. 3A(b) shows the case that the dorsal pancreatic artery branches from the celiac artery trunk, FIG. 3A(c) shows the case that the dorsal pancreatic artery branches from the common hepatic artery, FIG. 3B(d) shows the case that the dorsal pancreatic artery branches from the superior mesenteric artery, and FIG. 3B(e) shows the case that the dorsal pancreatic artery does not exist. The structure shown in FIG. 3A (a) is the most probable case for almost half of the human bodies. On the other hand, the structures shown in FIG. 3B (d) and FIG. 3B(e) are very rare cases. The embodiments 1 to 3 described below show the methods for forming effectively an enclosed area for the individual structures of the dorsal pancreatic artery. In order to understand straightforwardly the concept of the present invention, note that only a method for forming an enclosed area for the artery system depends upon the structures in the human body, and that a single method for forming an enclose area for the venous system can be applied commonly to all the human bodies, which leads to an identical structure of the enclosed area so formed.

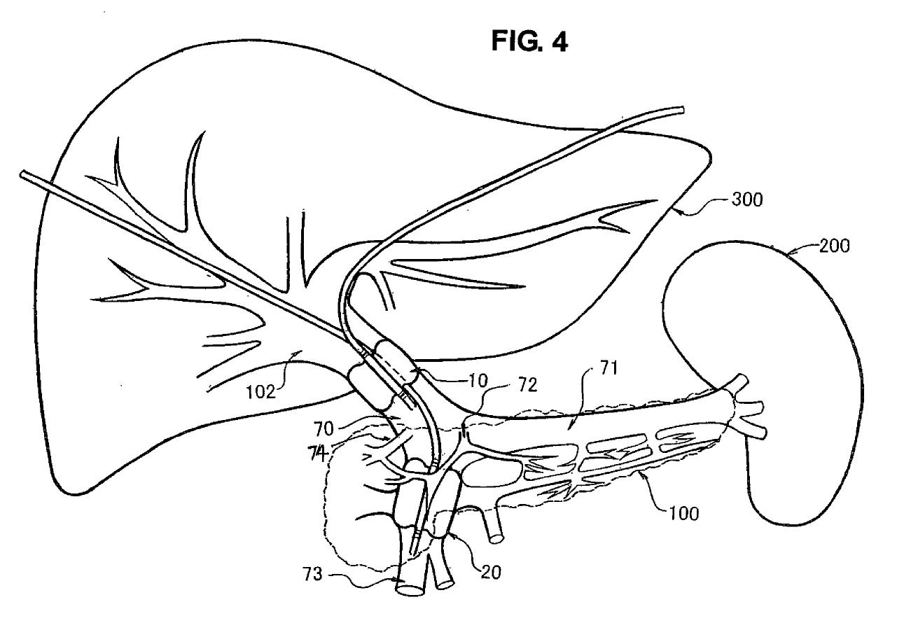

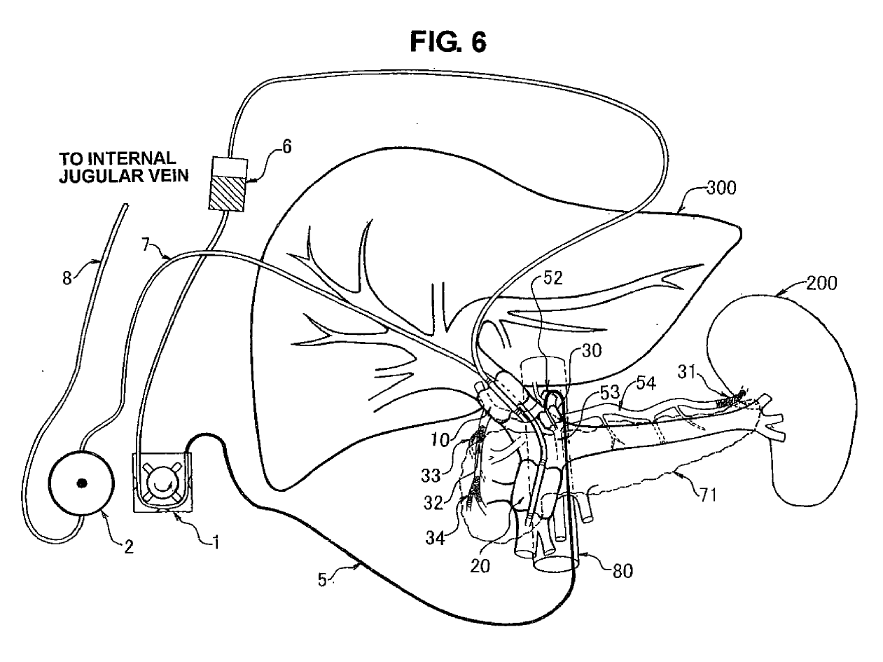

Next, a method of forming an enclosed area in the venous system is described by referring to FIG. 4. In order to understand easily the arrangement of occlusive balloon catheters, the shape of the balloon catheter is shown by solid lines in FIG. 4 and the rest of figures. In FIG. 4, components 10 and 20 are balloon catheters, respectively, and the component 300 is a lever. The other symbols in FIG. 4 represent the identical components as shown in FIG. 2. A couple of balloon catheters can be formed as a single unit of double balloon catheter. In operation, at first, the balloon catheter 20 may be inserted through the liver and the portal vein by way of percutaneous portal venous puncture, and then may be inflated and indwelled in the superior mesenteric vein 73 at the upstream of the confluence part of the superior mesenteric vein 73 and the splenic vein 71. Next in operation, the balloon catheter 10 may be inserted through the liver into the portal vein 70 in the similar manner to the balloon catheter 20, and then may be inflated and indwelled in the portal vein 70 at the downstream of the confluence part of the posterior pancreatic duodenal vein 74 and the portal vein 70. A couple of balloon catheters 10 and 20 have substantially an identical structure, and can suck a required amount of blood at the head of their catheter tubes. As for the balloon catheters 10 and 20, the balloon catheter disclosed in Japanese Patent Application Number 2006-357556 (2006) as described before is preferable with respect to the strength of the catheter tube and the expansion factor and the strength of the balloon. The operation after forming the enclosed area will be described in the embodiment referring to FIG. 6 and the subsequent figures.

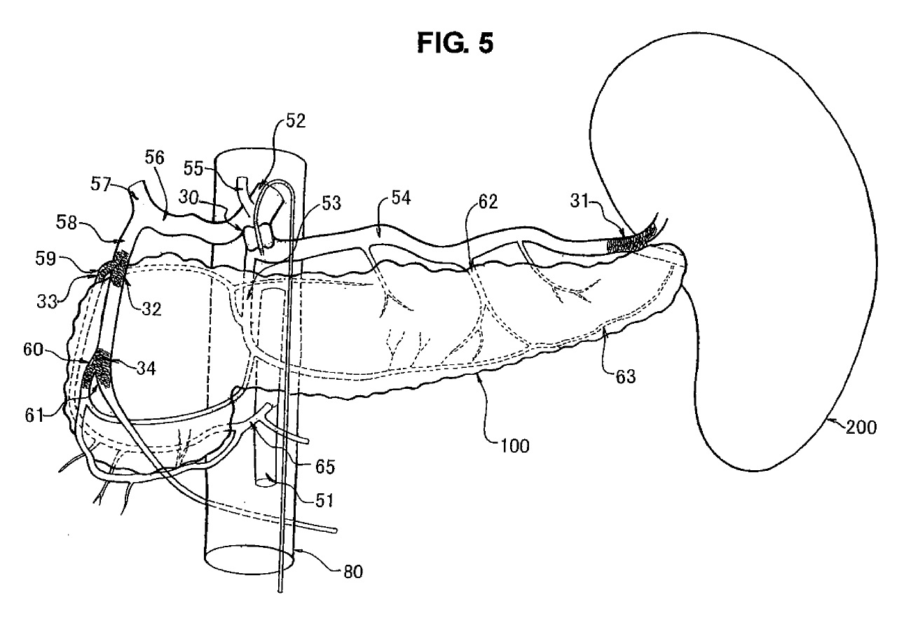

Next, a method for forming an enclosed area in the arterial system is described referring to FIG. 5. In FIG. 5, the component 30 is a balloon catheter and the components 31 to 334 are fine coils. The other symbols in FIG. 5 represent the identical components as shown in FIG. 1. FIG. 5 shows a method for forming an exemplary enclosed area for the most general structure as the arrangement of the human dorsal pancreatic artery. In operation, at first, in order to prevent the arterial flow from flowing into the spleen 200, plural coils 31 may be inserted one by one into the splenic artery 54 related to the spleen 200 from the aorta 50 through the celiac trunk 52 and the splenic artery 54. In the similar way, in order to prevent the arterial flow from flowing into the pancreas 100 from the superior mesenteric artery 51, the anterior superior pancreaticoduodenal artery 60 and the right gastro-omental artery 61 may be also occluded by coils as shown in the figure. Next in operation, the posterior superior pancreaticoduodenal artery 59 and the gastroduodenal artery 58 may be occluded by coils. Owing to those occlusions, the artery flow potentially allowed to the stomach and the duodenum (not shown) is substantially disconnected. At last in operation, the balloon catheter 30 may be inserted into the splenic artery 54 from the femoral region through the aorta 80 and the coeliac artery 52, and inflated inside the splenic artery 54 at the upstream of the dorsal pancreatic artery 53, and then the splenic artery 54 may be isolated fluidly from the coeliac artery 52 and the common hepatic artery 56. As the result of the above operations, the arterial system related to the pancreas can be substantially isolated from the other internal organs, and thus, an enclosed area can be formed. Note that the sequence of forming the occlusion parts in the blood vessels by using coils and balloons is not necessarily limited to the above described sequence. This means that the method for forming the enclosed part is independent of the present invention as long as the resultant enclosed area can be formed in the arterial system.

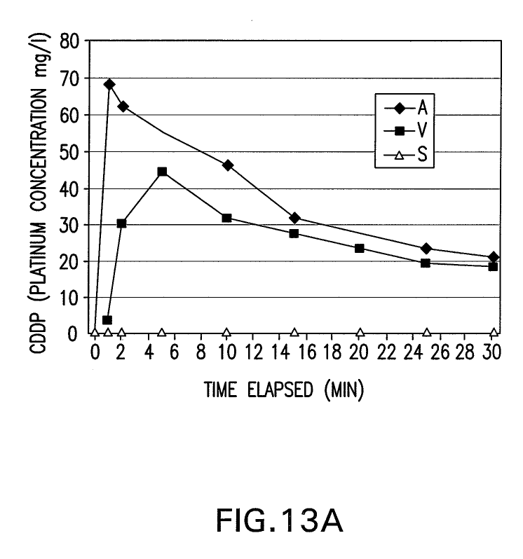

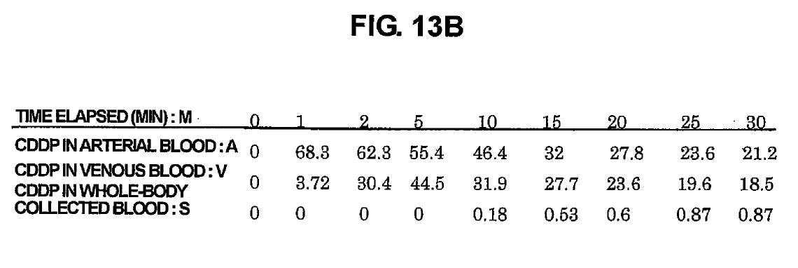

Some embodiments of the present invention will be described below by referring to FIGS. 6 to 13. FIGS. 6 to 12 show arrangements of the enclosed area related to the human pancreas and the balloon catheters. and FIGS. 13A and 13B show a graph and its related data used for describing the result in the embodiment 1, respectively.

### EMBODIMENT 1

FIG. 6 shows one exemplary structure of the perfusion system for the patient having the most general structure in which the dorsal pancreatic artery 53 branches from the splenic artery 54 as shown in FIG. 3A (a). In the embodiment of FIG. 6, for straightforward understanding of the perfusion system for pancreas treatment, the venous system shown in FIG. 4 and the arterial system shown in FIG. 5 are superposed visually. In FIG. 6, components 1 and 2 are perfusion pumps, and the component 6 is a reservoir for injecting the pharmaceutical such as anticancer agent. The indwelling method for the balloon catheters 10 and 20 in the venous system and the indwelling method for the balloon catheter 30 in the arterial system are as described in FIG. 4 and FIG. 5, respectively. In the subsequent embodiments, note that 10 Fr catheter is used for the balloon catheter 10 and 12 Fr catheter is used for the balloon catheters 20 and 30.

The venous blood inside the enclosed area may be sucked by the perfusion pump 1 at the head of the catheter tube of the balloon catheter 10 indwelled in the venous system. The pharmaceutical such as anticancer agent may be injected into the blood so sucked and extracted outside the human body at the reservoir 6. Next, the blood including the pharmaceutical may be supplied to the enclosed area in the arterial system through the catheter tube of the balloon catheter 30. The anticancer agent supplied to the enclosed in the arterial system contacts to the cancerous tumor (not shown) without substantially weeping out into the internal organs other than the pancreas or the other blood vessels, and then, flows into the enclosed area in the venous system. Next, the venous flow in the enclosed area in the venous system may be sucked again by the perfusion pump 1 at the head of the catheter tube of the balloon catheter 10 indwelled in the venous system. In order to reduce the pressure of the venous flow flowing into the superior mesenteric vein 73, the venous blood flowing into the superior mesenteric vein 73 may be sucked by the centrifugal pump 2 at the head of the catheter tube of the balloon catheter 20 as well as perfusion operation as described above. This venous blood is extracted outside the human body and then returned to the vein inside the human body through the internal jugular vein. In the above described treatment, the concentration of anticancer agent exposed to the tumor is 60 times as large as the concentration used in the ordinary treatment. Owing to this high concentration of anticancer agent, the tumor may be reduced or killed. As the amount of anticancer agent leaked outside from the enclosed area is as small as 1% or less of the injected anticancer agent, any bad effect to the other internal organs may be substantially negligible.

Now, the effect of the embodiment 1 is described by referring to FIG. 13A and FIG. 13B. FIG. 13A is a graph showing the platinum concentrations (mg/l) in the arterial blood, the venous blood and the whole-body collection of blood measured every 5 minutes, each collected in 30 minutes after starting the perfusion operation for the pharmaceutical such as anticancer agent. According to those measure data, it is proved that the pharmaceutical such as anticancer agent injected in the arterial blood effectively flows into the venous blood, and that the pharmaceutical injected in the arterial blood seldom or never leaks outside the enclosed area which is formed intentionally.

### EMBODIMENT 2

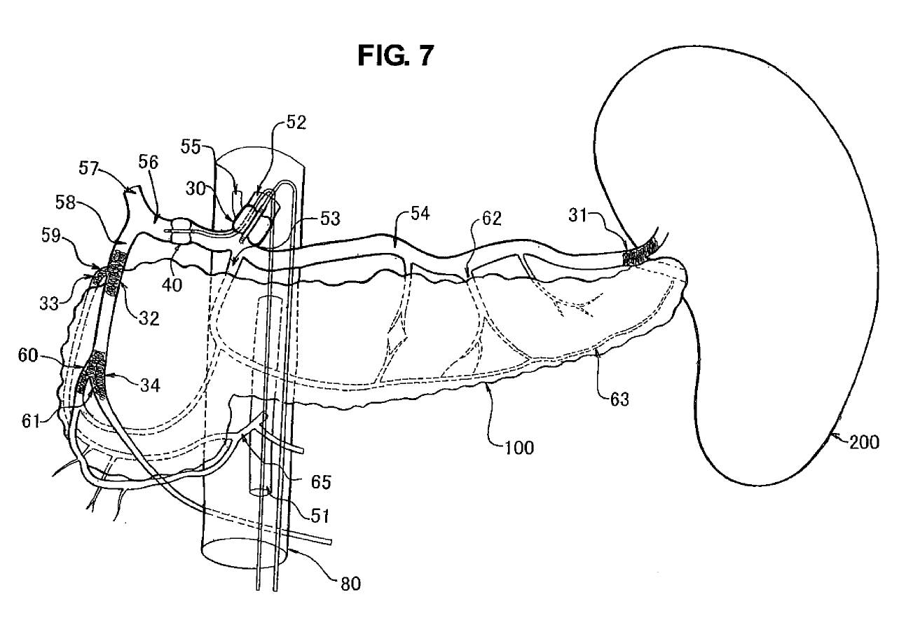

Next, refer to FIG. 7. FIG. 7 shows a method for forming the enclosed area in the arterial system in case that the dorsal pancreatic artery branches from the celiac artery trunk as shown in FIG. 3A (b). In this case, the occlusion position by using coils is the same as the case shown in FIG. 6, but a couple of balloon catheters 30 and 40 are used. At first, in similar matter to FIG. 6, the splenic artery 54 may be occluded by the coil 31. Next, the anterior superior pancreaticoduodenal artery 60 and the right gastro-omental artery 61 may be occluded by the coil 34. Next, the posterior superior pancreaticoduodenal artery 59 and the gastroduodenal artery 58 may be occluded by the coils 33 and 32, respectively. After completing the occlusion operation by way of coils, the balloon catheter 40 may be inserted through the aorta 80 and the coeliac artery 52 into the common hepatic artery 56 at first, and then inflated and indwelled there. Finally, the balloon catheter 30 may be inserted through the aorta 80 into the coeliac artery 52, and then, positioned in the coeliac artery 52 at the downstream of the bifurcation part to the left gastric artery 55, and inflated and indwelled there. Though the balloon catheter 30 is indwelled at the downstream of the bifurcation part from the coeliac artery 52 to the left gastric artery 55 in this embodiment, the balloon catheter 30 may be indwelled so as to close the coeliac artery 52 the case may be, which does not arise any problem.

After forming the enclosed area as described above, the pharmaceutical such as anticancer agent may be injected into the enclosed area in the arterial system through the catheter tube of the balloon catheter 30. The pharmaceutical such as anticancer agent injected into the enclosed area is provided mainly to the pancreas through the dorsal pancreatic artery 53. The balloon catheter 40 is provided in order to prevent the pharmaceutical such as anticancer from flowing through the proper hepatic artery 57 into the liver (not shown). As another feature of the occlusion operation in this embodiment is similar to the case shown in FIG. 6, its detail is not described here.

### EMBODIMENT 3

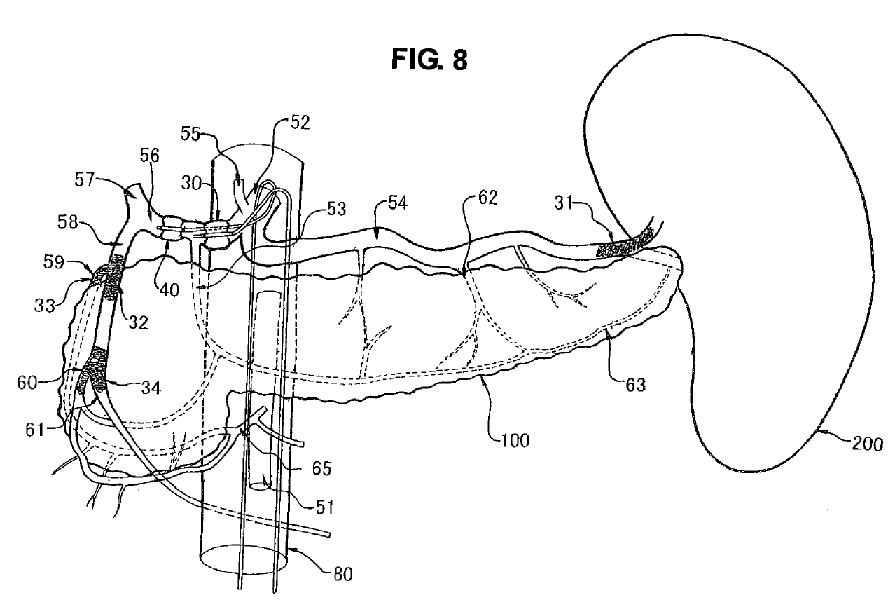

Next, refer to FIG. 8. FIG. 8 shows a method for forming the enclosed area in the arterial system in case that the dorsal pancreatic artery branches from the common hepatic artery as shown in FIG. 3A (c). In this case, the occlusion position by using coils is the same as the case shown in FIG. 6. This embodiment using a couple of balloon catheters is similar to the embodiment 2, but has different indwelling positions of the balloon catheters. The occlusion positions and occlusion method in this embodiment are the same as the embodiment 2. After completing the occlusion operation by way of coils, the balloon catheter 40 may be inserted through the aorta 80, the coeliac artery 52 and the common hepatic artery 56 into the proper hepatic artery at first, and then inflated and indwelled there. Finally, the balloon catheter 30 may be inserted through the aorta 80 and the coeliac artery 52 into the common hepatic artery 56, and then, positioned in the common hepatic artery 56 or the coeliac artery 52 at the upstream of the bifurcation part to the dorsal pancreatic artery 53, and inflated and indwelled there.

The pharmaceutical such as anticancer agent may be injected into the enclosed area in the arterial system through the catheter tube of the balloon catheter 30. The pharmaceutical such as anticancer agent injected into the enclosed area is provided mainly to the pancreas through the dorsal pancreatic artery 53. The balloon catheter 40 is provided in order to prevent the pharmaceutical such as anticancer from flowing through the proper hepatic artery 57 into the liver (not shown). As another feature of the occlusion operation in this embodiment is similar to the case shown in FIG. 6, its detail is not described here.

### EMBODIMENT 4

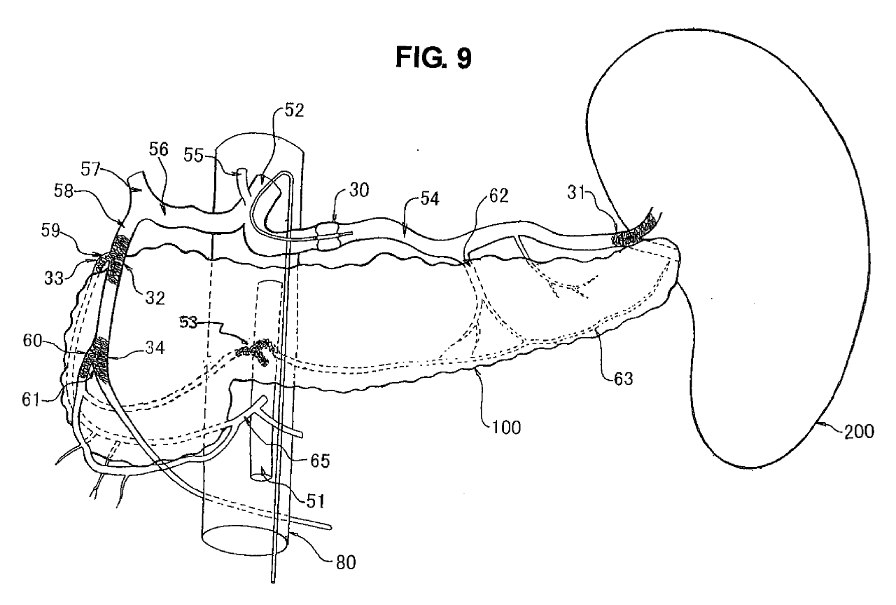

Next, refer to FIG. 9. FIG. 9 shows a method for forming the enclosed area in the arterial system in case that the dorsal pancreatic artery branches from the superior mesenteric artery as shown in FIG. 3A (d). In this case, in addition to the same occlusion position by using coils as the case shown in FIG. 6, the dorsal pancreatic artery 53 branching from the superior mesenteric artery 51 may be occluded by using coils as shown in the figure. Owing to this operation, the function of the dorsal pancreatic artery may be disabled. After completing the occlusion operation by way of coils, the balloon catheter 30 may be inserted into the splenic artery 54 in the similar manner to the embodiment 1, and then inflated and indwelled at an adequate position.

The pharmaceutical such as anticancer agent may be injected into the enclosed area in the arterial system through the catheter tube of the balloon catheter 30. The pharmaceutical such as anticancer agent injected into the enclosed area is provided mainly to the pancreas in the similar manner to the embodiment 1. As the subsequent occlusion operation in this embodiment is similar to the case shown in FIG. 6, its detail is not described here.

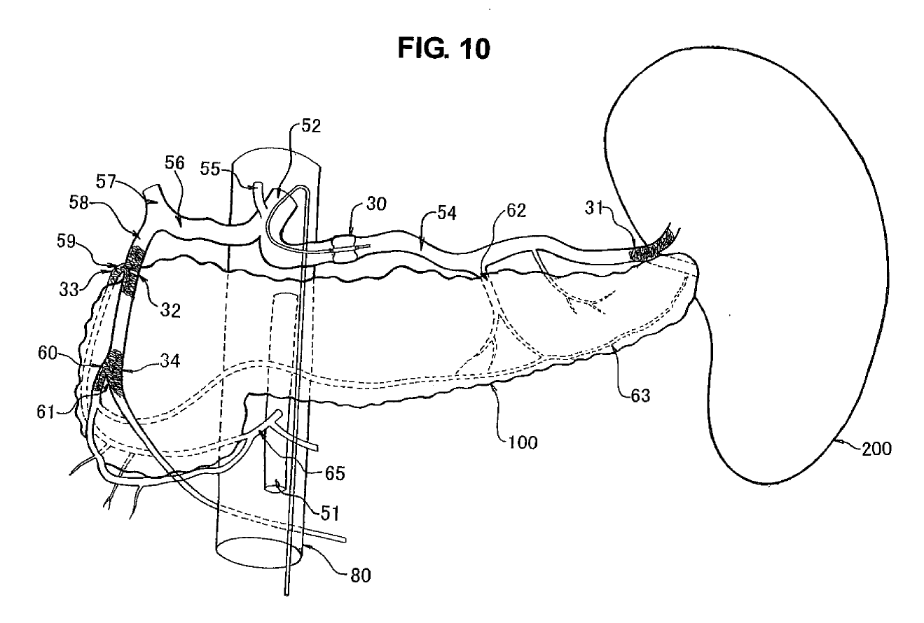

Next, refer to FIG. 10. FIG. 10 shows a method for forming the enclosed area in the arterial system in case that there is no dorsal pancreatic artery. In this embodiment, the occlusion positions by using coils are the same as the case shown in FIG. 6. As the indwelling positions of the balloon catheters and their operation are the same as the embodiment 4 shown in FIG. 9, its detail is not described here. It will be appreciated that the perfusion system according to this embodiment can be applied directly to the case in which the dorsal pancreatic artery 53 has been invaded by cancer cells.

### EMBODIMENT 6

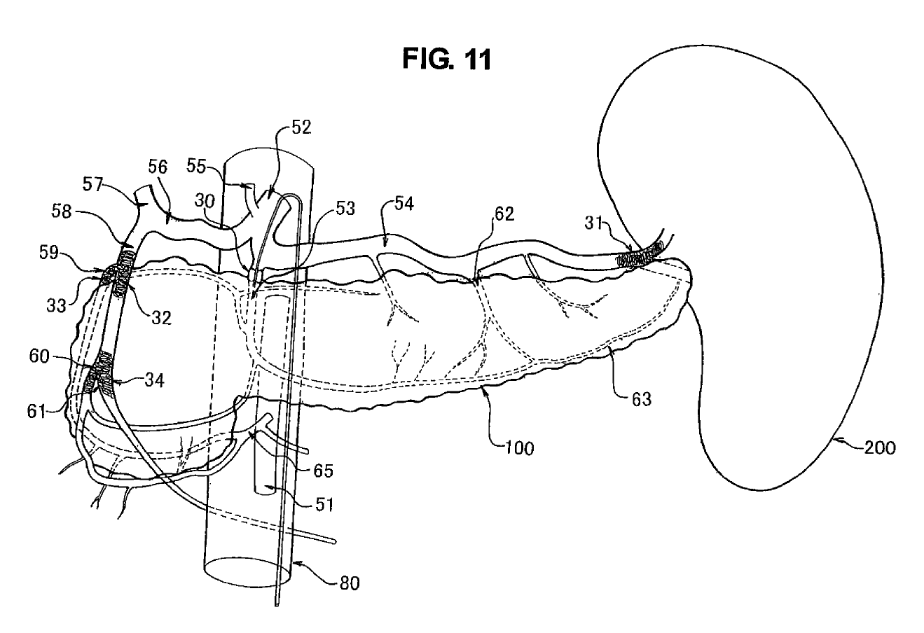

Next, refer to FIG. 11. FIG. 11 shows a method for forming the enclosed area in the arterial system in case that micro balloon catheter is used. Though the dorsal pancreatic artery branches from the splenic artery as shown in FIG. 11, the enclosed area in the arterial system can be formed in the similar manner to the case shown in FIG. 11 even if the dorsal pancreatic artery may has a different structure from those shown in FIG. 3A. As for the micro balloon catheter used in this embodiment, 3 Fr balloon catheter is preferable. Note that it is obvious that normal balloon catheters rather than micro balloon catheters may be applied to the patient whose dorsal pancreatic artery 53 has an enough diameter.

### EMBODIMENT 7

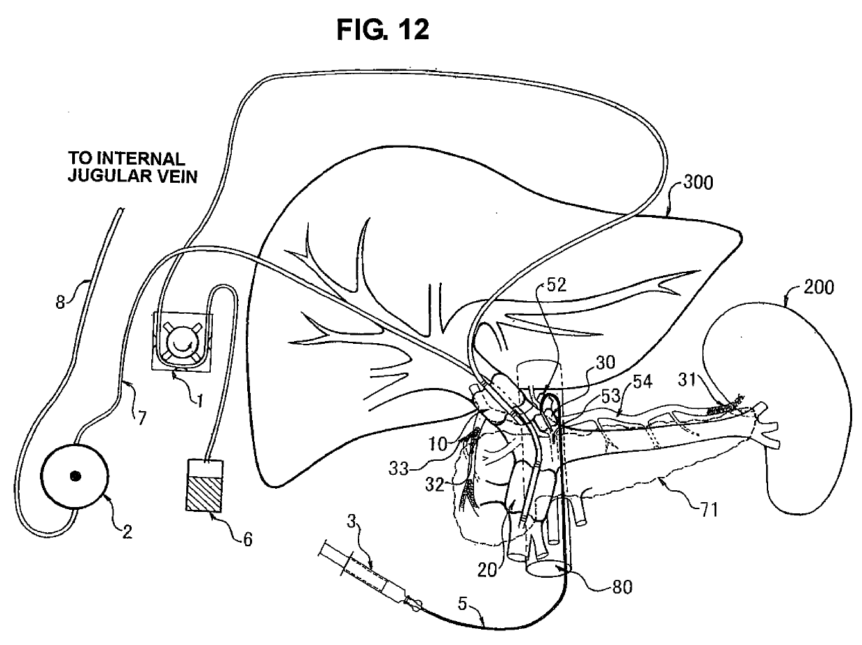

Embodiments 1 through 5 use a relatively large-scale perfusion system so that the blood inside the closed venous system may be extracted outside the human body, and may be returned to the closed arterial system after applying a necessary process to the extracted blood. It is allowed that the pharmaceutical can be injected from the syringe apparatus such as injection syringe for the simplified treatment. FIG. 12 shows an exemplary structure of the perfusion system for pancreas treatment in case of applying the syringe apparatus. The basic structure of the perfusion system for pancreas treatment shown in FIG. 12 is almost the same as the basic structure of the perfusion system of the embodiment 1. The perfusion system shown in FIG. 12, in stead of using “a perfusion apparatus for extracting the blood in the human body outside the human body, injecting a pharmaceutical into the extracted blood and then perfusing the blood into the human body”, is so configured that “a pump apparatus for sucking the blood inside the human body outside the human body and for returning the sucked blood into the reservoir and a syringe apparatus for injecting the pharmaceutical into the enclosed area” is used and that the pharmaceutical may be injected through the third balloon catheter into the artery flow inside the enclosed area by the syringe apparatus. The amount of the pharmaceutical injected by the syringe apparatus is preferably set to about 30 to 60 cc per minute and its injection preferably continues for 20 to 40 minutes. The blood temporally stored in the reservoir may be disposed after the treatment.

Though some embodiments according to the present invention have been described by referring to corresponding figures as above, the present invention is not limited to those embodiments but should be interpreted in the spirit of the invention. For example, though some locations are occluded by using the coil apparatus in the above described embodiments, it is allowed that those locations may be occluded by using narrow and elongated balloons.

The specification of the present invention uses, as a bibliography, “Pictorial book for X-ray anatomy of abdominal blood vessels” edited by Dr. Kyouichi Hiramatsu, published by IGAKU-SHOIN Ltd. Tokyo, Japan, in Sep. 1, 1991.

The present invention is obviously effective for the treatment of pancreatic cancer as described above. It will be appreciated that the perfusion system according to the present invention may be effectively applied to the genetic therapy and the treatment of sugar diabetes by using the gene-therapeutic agent and other pharmaceutical agent in stead of using the anticancer agent for the pharmaceutical.

What is claimed is:

1. A perfusion system for pancreas treatment comprising;
an arterial system occluding means for occluding a main blood vessel of an arterial system so that a blood in said arterial system related to a pancreas may not be substantially flow into an internal organ other than the pancreas, and forming an enclosed area in said arterial system related to the pancreas;
a venous system occluding means for occluding a main blood vessel of a venous system so that a blood in said venous system related to the pancreas may not substantially flow into an internal organ other than the pancreas, and forming an enclosed area in said venous system related to the pancreas;
a perfusion apparatus for extracting the blood inside the enclosed area of said venous system outside a human body, injecting a pharmaceutical into the extracted blood and then perfusing said venous blood into the enclosed area of said artery system; and
a means for extracting the venous blood flow which is flowing into the enclosed area of said venous system outside the human body, and re-circulating the extracted venous blood flow into the human body at another part of the human body, wherein
a recirculation of the blood into the enclosed area of said arterial system may be performed by said arterial system occluding means, and a recirculation of the blood from the enclosed area of said venous system may be performed by a venous system occluding means.

2. The perfusion system for pancreas treatment of claim 1, wherein
said arterial system occluding means is configured with at least one balloon catheter and plural coil apparatus; and
said venous system occluding means is configured with a couple of balloon catheters.

3. A perfusion system comprising;
a first balloon catheter provided at a downstream side of a confluence part where a blood flow of a splenic vein flows in a portal vein so as to occlude said portal vein;
a second balloon catheter provided at an upstream side of said confluence part so as to occlude a superior mesenteric vein;
a third balloon catheter provided at a downstream side of a bifurcation part where a blood flow in an abdominal aorta flows into a splenic artery and at an upstream side of a bifurcation part where a blood flow from said splenic artery flows into a dorsal pancreatic artery so as to occlude said splenic artery;
a first occlusion device provided at a upstream side where said splenic artery branches at a pancreatic branch of splenic artery so as to occlude said splenic artery in order to prevent a blood flow in said splenic artery from flowing into a spleen;
a second occlusion device for preventing a relatively large amount of arterial flow from flowing into a stomach and a duodenum; and
a perfusion apparatus for extracting a venous blood inside a human body, injecting a pharmaceutical into the extracted venous blood and then perfusion said venous blood into the human body, wherein
the venous flow occluded inside an enclosed area by above described plural occlusion device is further extracted through said first balloon catheter outside the human body, and then the extracted venous blood including a pharmaceutical is returned through said third balloon catheter again to the artery inside the enclosed area; and
the blood inside said superior mesenteric vein is extracted outside the human body through said second balloon catheter at an upstream of an indwelling position of said second balloon catheter, and then the extracted blood is returned to the human body again at another position of the human body.

4. The perfusion system of claim 3, wherein
said first and second occlusion device are coil apparatus, respectively, for occluding a blood vessel by plural coils.

5. The perfusion system of claim 1, wherein
said first and second occlusion balloon catheters are formed as a single double-balloon catheter having at least one blood intake port provided between a couple of balloons mounted at a catheter tube; and
the blood inside said closed area is extracted through said intake port outside the human body, and, after injecting a pharmaceutical into the extracted blood, the extracted blood is returned through said third catheter to the human body again.

6. A perfusion system comprising;
a first balloon catheter provided at a downstream side of a confluence part where a blood flow of a splenic vein flows in a portal vein so as to occlude said portal vein;
a second balloon catheter provided at an upstream side of said confluence part so as to occlude a superior mesenteric vein;
a third balloon catheter provided so as to occlude a dorsal pancreatic artery;
a first occlusion device provided at a downstream side where said splenic artery branches at a pancreatic branch of splenic artery so as to occlude said splenic artery in order to prevent a blood flow in said splenic artery from flowing into a spleen;
a second occlusion device for preventing a relatively large amount of arterial flow from flowing into a stomach and a duodenum; and
a perfusion apparatus for extracting a venous blood in a human body outside the human body, and then, after injecting a pharmaceutical into the extracted venous blood, perfusing the venous blood into the human body again, wherein
the venous blood occluded inside the enclosed area by said plural occlusion device is extracted through said first balloon catheter outside the human body, and then, the extracted venous blood including a pharmaceutical is returned through said third balloon catheter again to an artery inside said enclosed area by said perfusion apparatus; and
the blood inside said superior mesenteric vein is extracted outside the human body through said second balloon catheter at a downstream of an indwelling position of said second balloon catheter, and then the extracted blood is returned to the human body again at another position.

7. A perfusion systems for pancreas treatment comprising;
a first balloon catheter provided at a downstream side of a confluence part where a blood flow of a splenic vein flows in a portal vein so as to occlude said portal vein;
a second balloon catheter provided at an upstream side of said confluence part so as to occlude a superior mesenteric vein;
a third balloon catheter provided at a downstream side of a bifurcation part where a blood flow in an abdominal aorta flows into a splenic artery and at an upstream side of a bifurcation part where a blood flow from said splenic artery flows into a dorsal pancreatic artery so as to occlude said splenic artery;
a first occlusion device provided at a downstream side where said splenic artery branches at a pancreatic branch of splenic artery so as to occlude said splenic artery in order to prevent a blood flow in said splenic artery from flowing into a spleen;
at least one, second occlusion device provided so as to occlude a blood flow in a gastroduodenal artery and a posterior superior pancreaticoduodenal artery;
a pump apparatus for sucking a blood inside a human body outside the human body and for returning the sucked blood into the human body again; and
a syringe apparatus for injecting a pharmaceutical into said enclosed area, wherein
the pharmaceutical is injected into an arterial flow inside said enclosed area through said third balloon catheter by said syringe apparatus; and
a blood inside said superior mesenteric vein is extracted outside the human body through said second balloon catheter at a downstream of an indwelling position of said second balloon catheter, and then the extracted blood is returned to the human body again at another position of the human body.
